(19) United States
(12) Patent Application Publication
(10) Pub. No.: US 2008/0188573 A1
(43) Pub. Date: Aug. 7, 2008

(54) USE OF THY1-FP TRANSGENIC MOUSE FOR THE IDENTIFICATION OF OPHTHALMIC AGENTS

(75) Inventors: Ji-Ye WEI, Colleyville, TX (US); Richard L. ORNBERG, Burleson, TX (US); Gustav GRAFF, Cleburne, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/025,515

(22) Filed: Feb. 4, 2008

Related U.S. Application Data
(60) Provisional application No. 60/887,938, filed on Feb. 2, 2007.

(51) Int. Cl. A61K 45/00; G01N 33/00

(52) U.S. Cl. 514/789; 800/3

(57) ABSTRACT

The present invention relates to the use of thy1-FP (fluorescent protein) non-human transgenic animals such as mice to screen for ophthalmic agents that can be used to treat corneal dry eye syndrome and/or retinal neuropathies associated with glaucoma and/or age-related macular degeneration. Such ophthalmic agents may be selected by assessing their ability to provide protection in ocular tissue.

### CROSS-REFERENCE TO RELATED APPLICATION

This application claims priority under 35 U.S.C. §119 to U.S. Provisional Patent Application No. 60/887,938 filed Feb. 2, 2007, the entire contents of which are incorporated herein by reference.

### TECHNICAL FIELD

The present invention relates to the fields of cell and molecular biology, pharmacology and medicine. More specifically, the present invention relates to the use of thy1-FP (fluorescent protein) transgenic mice to screen for ophthalmic agents that can be used to treat corneal dry eye syndrome and/or retinal neuropathy disorders, associated with glaucoma and age-related macular degeneration (AMD).

### BACKGROUND OF THE INVENTION

The discovery and development of modern ophthalmic agents requires a detailed understanding of the functional neuroanatomy of the eye and the neuropathology associated with ophthalmic diseases. There are two major neural systems within the eye. One system innervates the cornea and ciliary body, and the other innervates the retina. The cornea is the most innervated tissue of the body and is supplied by sensory and autonomic nerves that provide protective and trophic functions for corneal repair after corneal disease, trauma or surgery. Recently, corneal innervation has gained attention due to dramatic changes in the morphology of the corneal nerve bed in patients with diabetes, Sjogren's syndrome, and dry eye. See, e.g., Quadrado et al., Cornea, Vol. 25:761-8, 2006. The retina contains the neuronal elements that transduce light and process image information that is transmitted to the brain for interpretation. Structural changes in the retina associated with glaucoma and AMD are a critical part of diagnosing and treating such retinal diseases.

Preclinical testing and development of ophthalmic agents requires extensive testing or screening in appropriate animal models of each disease state; accordingly several models for corneal dry eye, glaucoma and AMD have been developed. However, anatomical animals models used to assess therapeutic efficacy are lacking. For example, there is no model that allows either the in vivo or flat mount imaging of the entire corneal neuronal sensory network in experimental animals. Similarly, there are no models that image, either in vivo or ex vivo, all retinal neurons before and after treatment with test agents. Embodiments of the present invention provide, for the first time direct and three-dimensional images of the entire corneal sensory nerve network and the retinal nerve network in ex vivo tissue whole mounts without the application of histological/immunocytochemical staining and/or invasive in vivo labeling procedures.

### BRIEF SUMMARY OF THE INVENTION

The present invention relates to the use of non-human transgenic animals in order to obtain an image directly in live animals of the entire corneal sensory nerve and retinal nerve network. Further, the animals of the present invention can be used to perform ex vivo tissue whole mounts without the application of histological/immunocytochemical staining and/or invasive in vivo labeling procedures. Thus, these animals can be used to easily assess the safety and efficacy of potential therapeutics in vivo. Yet further, these animals can be used to determine agents that induce and/or cause inflammation in the cornea and/or retinal tissue and to assess the effects of such stimuli on the neuronal network.

One embodiment of the present invention comprises a method of manufacturing a topical ophthalmic agent comprising the steps of: (a) providing a non-human transgenic animal that selectively expresses a fluorescent protein (FP) in ocular tissue; (b) providing a candidate substance suspected of providing protection to ocular nervous tissue (e.g., corneal and/or retinal tissue); (c) administering the candidate substance to the animal; (d) selecting the agent by assessing the ability of the candidate substance to provide protection in ocular nervous tissue; and (e) manufacturing the selected ophthalmic agent. The FP is green fluorescent protein (GFP) or yellow fluorescent protein (YFP) and the FP expression is driven by a neuron-specific promoter, for example, thy-1.

In certain embodiments, protection can be determined by measuring a decrease in the degeneration of ocular related neurons, such as retinal ganglion or optic nerve axons. Such an agent could be used to treat a subject suspected of or suffering from glaucoma or AMD. Protection can also be determined by measuring the formation of an abnormal corneal nervous system, or an increase in tear film secretion, a decrease in tear film break up time, etc. Such an agent could be used to treat a subject suspected of or suffering from corneal dry eye syndrome.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. Figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawing and wherein:

FIG. 1A and FIG. 1B show fluorescent microscope images of corneal nerve fibers of the naive thy-1 FP transgenic mouse expressing GFP in upper sub-epithelial regions (FIG. 1A) and lower subepithelial regions (FIG. 1B).

FIG. 2A and FIG. 2B show fluorescent images of retinal ganglion cell bodies and nerve fibers in Thy1-GFP transgenic mouse eye. FIG. 2A shows a low magnification view illustrating punctate labeling of ganglion cell bodies and radial streaks of nerve fibers eminating from the optic nerve (black hole in lower right). FIG. 2B shows a high magnification view of retinal ganglion cell bodies and overlying bundles nerve fibers that coalesce at the optic nerve head to from the optic nerve, cranial nerve II.

FIG. 3A, FIG. 3B and FIG. 3C show fluorescent images of retinal ganglion cell bodies. FIG. 3A is two dimension maximum project view of stack of images collected from the retinal surface down to the start of the amacrine layer. A more three dimensional view, (FIG. 3B), shows that the cells of the vasculature such as the endothelium, pericytes or smooth muscle do not express GFP. Cross section representations shown in FIG. 3C, reveal processes between the RGC and amacrine cells of the naive animal.

### DETAILED DESCRIPTION OF THE INVENTION

### I. DEFINITIONS

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

As used herein, the term “dry eyes” or “dry eye syndrome” refers to a condition in which there is a lack of sufficient lubrication and/or moisture in the eye or inadequate tear production and an associated increase in corneal sensitivity (i.e., pain). Dry eye syndrome is also called keratoconjunctivitis sicca. Dry eye disorder or dry eye syndrome can be caused from decreased production of fluids from the tear glands and/or an imbalance in the formation or composition of the produced tears. Any method known to those of ordinary skill in the art can be used to diagnosis dry eye syndrome. For example, diagnosis can be made by obtaining a history from a subject, including a history of symptoms such as burning, itching, irritation, redness, blurred vision that improves with blinking, or increased discomfort after a period of reading. Diagnosis may also be by clinical examination, such as by slit lamp examination to assess the tear film and to evaluate the subject for corneal findings that may be associated with dry eye syndrome. Diagnosis may also be by measurement of tear production. In some embodiments, diagnosis is obtained by measuring a five-minute Schirmer's test with or without anesthesia using a Whatman #41 filter paper 5 mm wide by 35 mm long. Less than 5 mm wetting of the filter paper with anesthesia and/or less than 10 mm wetting without anesthesia is indicative of dry eye syndrome. Other methods to diagnose dry eye syndrome include measurement of a tear break-up time, a tear protein analysis, or a lactoferrin analysis. A combination of any of these methods may be used to diagnosis dry eye syndrome.

As used herein, the term “corneal inflammation” refers to a condition in which the corneal tissue is irritated and/or red. Typically, inflammation of the cornea commonly presents as a painful red eye with reduced visual acuity due to cellular infiltration and possibly corneal edema.

As used herein, the term “fluorescent protein” or “FP” refers to proteins that produce chromophores; for example, the chromophores can be produced via cyclization and oxidation of the protein. Those of skill in the art understand the fluorescent protein or FP can include but are not limited to red (RFP), green (GFP), yellow (YFP) or cyan (CFP) fluorescent proteins. Yet further, other FPs can include variants of RFP, GFP, YFP or CFP; for example enhanced green fluorescent protein (EGFP).

As used herein, the term “intraocular pressure” or “IOP” refers to the pressure of the fluid inside the eye. In a normal human eye, IOP is typically in the range of 10 to 21 mm Hg. IOP varies among individuals; for example, it may become elevated due to anatomical problems, inflammation of the eye, as a side-effect from medication or due to genetic factors. “Elevated” intraocular pressure is usually considered to be ≧21 mm Hg, which is also considered to be a risk factor for the development of glaucoma. However, some individuals with an elevated IOP may not develop glaucoma and are considered to have ocular hypertension.

As used herein, the terms “glaucomatous optic neuropathy” or “glaucoma” are interchangeable. Glaucoma refers to a disease characterized by the permanent loss of visual function due to irreversible damage to the optic nerve. The two main types of glaucoma are primary open angle glaucoma (POAG) and angle closure glaucoma.

As used herein, the term “ocular nervous tissue” refers to neuronal tissue or nerves associated with ocular tissue, for example the retina and cornea.

As used herein, the term “optic nerve” refers to the nerve or cranial nerve II, which transmits visual information from the retina to the brain.

As used herein, the term “optic nerve damage” refers to an alteration of the normal structure or function of the optic nerve. The alteration of the normal structure or function of the optic nerve may be the result of any disease, disorder or insult. Such diseases or disorders include, but are not limited to optic neuropathies (e.g., ischemic neuropathies) or glaucoma. An alteration of the normal function of the optic nerve includes any alteration of the ability of the optic nerve to function appropriately, such as transmit visual information from the retina to the brain. An alteration in function may manifest itself, for example, as loss of visual field, impaired central visual acuity, abnormal color vision, and so forth. Examples of alteration of structure include nerve fiber loss in the retina, abnormal cupping of the optic nerve and pallor of the optic, swelling of the optic nerve. “Optic nerve damage” as used herein may include optic nerve damage to one or both optic nerves of a subject.

As used herein, the term “macular degeneration” refers to a disease associated with deterioration of the structure and/or function of the macula. Macular degeneration may occur at any age. Causative factors include genetic factors and environmental factors. Examples of types of macular degeneration include age-related macular degeneration, Stargardt's disease, and myopic degeneration.

As used herein, the term “age-related macular degeneration” is characterized by loss of central vision in one or both eyes as a result of deterioration of the macula, which typically occurs in elderly individuals. There are two types of macular degeneration: “wet” (disciform or exudative) and “dry” (atrophic). The dry form of AMD is associated with the presence of drusen and/or retinal pigment epithelial changes in the macula region. Exudative age-related macular degeneration is associated with the presence of choroidal neovascularization in a subretinal location, which may eventually result in permanent vision loss due to scar tissue formation under the macula.

As used herein, the term “retinal neuropathy” is characterized as a loss of function or degeneration in the retinal nerves, for example degeneration or loss of retinal ganglion cells. Loss of function and/or degeneration of retinal nerves can be associated with a variety of ocular diseases and/or disorders such as, but not limited to glaucoma and AMD.

As used herein, the term “protection to ocular tissues” is characterized by assessing the beneficial effect of the agent using known parameters compared to a control subject that did not receive the agent. For example, to determine the protection of an agent for corneal dry eye syndrome, one can measure tear film secretion and tear film break up time, measure the chemical composition of tear film and perform ocular surface evaluation with fluorescein, lissamine green or rose bengal stain. Yet further, confocal scanning laser opthalmoscopy can also be used to assess the agents in vivo. To determination the protection of an agent for retinal neuropathies associated with glaucoma and AMD, one can assess therapeutic efficacy by decreasing IOP, reducing the loss of retinal ganglion cells and/or the loss of optic nerve axons.

As used herein, the term “control subject” is characterized by having similar symptoms and/or disease and/or disorder as the test subject, however, the control subject does not receive any of the test agent or candidate agent or substance. A control subject or test subject can also be characterized by having one eye serve as a test subject and the contralateral eye serve as a control.

As used herein, the term “susceptibility of dry eyes” or “suspected of having dry eye” or “at risk for developing dry eye” refers to an individual or subject that is likely to develop or have dry eyes. For example, a subject that is susceptible to dry eyes may be a subject suffering from menopause, a subject that lives in a hot, dry or windy climate, a subject exposed to high altitudes, a subject that spends excessive amounts of time reading and/or working on a computer, a subject that wears contact lens, a subject having undergone surgery (e.g., LASIK), a subject exposed to air conditioning and/or heating, a subject suffering from thyroid conditions, vitamin A deficiency, Parkinson's, diabetes, rheumatoid arthritis, SLE, and/or Sjogren's, a subject suffering from eyelid disorders (e.g., blepharitis, Bell's palsy, facial palsy, Grave's disease) and a subject suffering from side effects from drugs, for example antihistamines, decongestants, antidepressants, diuretics, beta blockers, oral contraceptives, opiate-based drugs, THC-based drugs.

As used herein, the terms “susceptible to glaucoma,” or “susceptibility to developing glaucoma” refers to an individual or subject that is at risk of developing glaucoma. For example, the subject may have elevated intraocular pressure in one or both eyes without any other findings associated with glaucoma. While such an individual does not clinically carry a diagnosis of glaucoma, such an individual is at risk of developing glaucoma by virtue of the presence of the elevation in intraocular pressure. For example, the intraocular pressure may be greater or equal to 21 mm Hg in one or both eyes. A subject without elevated intraocular pressure who does not have glaucoma may also be susceptible to the development of glaucoma. For example, the subject may have a family history of glaucoma. The subject may or may not have a family history of glaucoma. “Susceptibility” is determined and assessed by any method known to those of ordinary skill in the art. For example, susceptibility can be determined based on results of physical examination, family history, or genetic screening techniques well-known to those of ordinary skill in the art.

As used herein, the term “susceptibility to retinal neuropathy” or “suspected of having retinal neuropathy” refers to an individual or subject that is likely to develop or at risk of having degeneration of retinal nerves or degeneration and/or loss of retinal ganglion cells. The subject may or may not have a family history of retinal neuropathies. “Susceptibility” is determined and assessed by any method known to those of ordinary skill in the art. For example, susceptibility can be determined based on results of physical examination, family history, or genetic screening techniques well-known to those of ordinary skill in the art. For example, an individual may be at risk of developing age-related macular degeneration if that individual is elderly and/or has a family history of age-related macular degeneration.

The terms “treatment” and “treating” refer to administration or application of a therapeutic agent to a subject or performance of a procedure or modality on a subject for the purpose of obtaining a therapeutic benefit of a disease or health-related condition. Thus, one of skill in the art realizes that a treatment may improve the disease condition, but may not be a complete cure for the disease.

### II. FP TRANSGENIC MICE

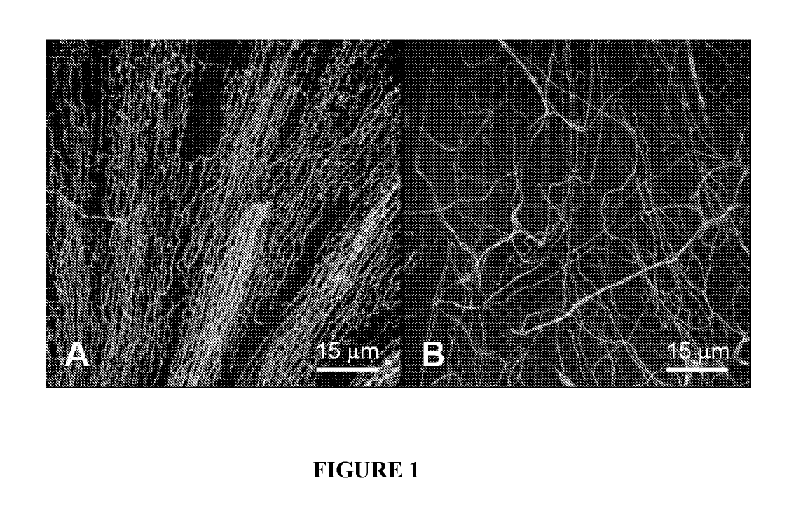

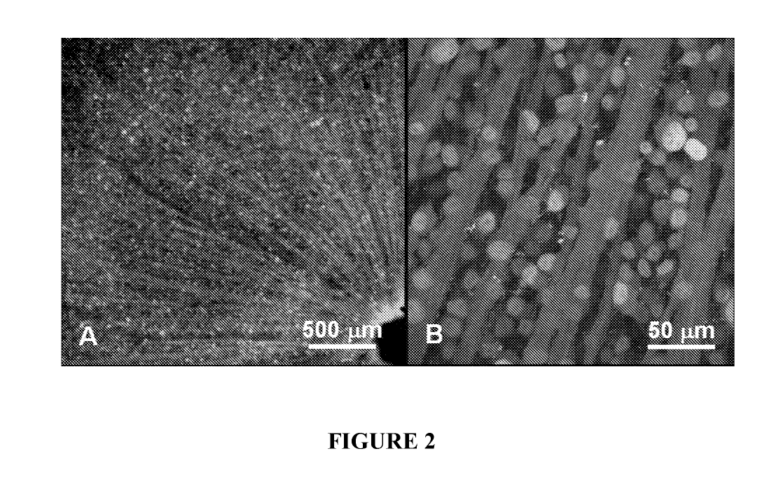

In a search for simpler and noninvasive methods for identifying and monitoring the corneal sensory nerves, FIG. 1, and retinal ganglion cell neurons in situ, FIG. 2, the inventors utilize transgenic mice expressing a FP under the control of the mouse thy1.2 promoter. Once such mouse that can be employed is the thy1-GFP mouse produced by Feng et al. (Neuron Vol. 28:41-51, 2000). Another mouse that may be used is available through The Jackson Laboratory, Bar Harbor, Me., under Strain Name: B6.Cg-Tg(Thy1-YFP)16Jrs/J; Stock No: 003709.

A. Fluorescent Proteins

The green fluorescent protein (GFP), a single peptide of 238 amino acids derived from the jellyfish Aequorea victoria, absorbs blue light and emits green light without a requirement for any cofactor or substrate. After the formation of its fluorophore by endogenous posttranslational cyclization, GFP is quite stable and remains fluorescent even after the harsh treatments found in many biochemical assays, such as 1% sodium dodecyl sulphate (SDS), 4% formaldehyde, and incubation gat 65° C. Since the first report of its use in Escherichia coli and Caenorhabditis elegans by Chalfie et al. (1994), GFP has found many applications as a reporter gene in a number of higher organisms including Drosophila (Wang et al., 1994) and zebrafish (Amsterdam et al., 1995; Peters et al., 1995).

Fluorescent proteins, such as GFP, can render cells fluorescent by introduction of cDNA encoding the protein itself. Thus, FPs can be detected in cells and tissues without having to add cofactors or substrates. Also, they are extremely stable, allowing their fluorescence to be monitored over extended periods. Those of skill in the art realize that any commercially available fluorescent protein can be used to produce transgenic mice. Examples of such FPs include, but are not limited to GFP, YFP, RFP and CFP. Those of skill in the art realize that these proteins can be altered to enhance the fluorescence, etc., thus, these variants may also be included, for example, ECFP, EGFP, EYFP or DsRed1. Commercial manufacturers of FPs include, Clontech Laboratories, Inc., Mountain View, Calif. that provides Living Colors® Fluorescent Proteins; Evrogen Joint Stock Company, Moscow, Russia. Vivid Colors™ vectors (Emerald Green (EmGFP) and Yellow (YFP) Fluorescent Protein) are also available from Invitrogen™.

The versatility of the GFP is enhanced by its ability to remain fluorescent as a fusion protein allowing studies of the subcellular distribution and dynamics of various proteins, including NMDA receptors (Marshall et al., 1995; Niswender et al., 1995; Aoki et al., 1996). Recently, a “humanized” version of GFP has become available in which silent mutations were introduced to alter the codons to those more commonly used in mammals. The “humanized” GFP is generally expressed at higher levels in mammalian systems than wild-type GFP. Also, mutant forms of GFP have become available which emit light of greater intensity or which exhibit wavelength shifts. (See Clontech Catalogue, 1998).

Thy1 gene is about an 8 kb genome fragment that is expressed in numerous cell types, including thymocytes, peripheral T cells, and neurons (Morris, 1985). The thy1 gene can be altered to force primarily expression in neuronal cells by removing the sequences in introns 3 and 4 (Vidal et al., 1990; Kelley et al., 1994; and Caroni, 1997). Thus, GFP when expressed in transgenic mice under the control of the thy1 gene lacking the sequences in introns 3 and 4, efficiently labels ocular nervous tissue. The intensity of the fluorescent signal, and the simplicity of the assay system (observation with standard fluorescein fluorescence optics), make GFP the reporter gene of choice for certain embodiments of the present invention.

B. Transgenic Mice as Models

The fluorescent signal generated by the FP in the mice of the present invention is strong enough that it is readily visible in at least two sites amenable to imaging of live animals, the retina and cornea. The retina is an extension of the central nervous system, and contains photoreceptors, amacrine, bipolar, and retina ganglion neurons, astrocytes in the nerve fiber layer and the astrocyte-like Mueller cells that span all retinal layers. Recent studies by Sabel et al. (1997), demonstrate that minor modifications to the optics of a standard confocal microscope allowed visualization, in a living rat, of retinal neurons that were labeled with fluorescent markers such as FluoroGold® by retrograde transport (Naumann et al., 2000).

The cornea is an epithelial surface that is highly innervated by sensory nerve fibers from the ophthalmic branch of the trigeminal nerve. As these nerve fibers pass through the corneal stroma, a structure that accounts for most of the corneal thickness, the fibers are ensheathed by non-myelinating Schwann cells. Slit-lamp confocal microscopes suitable for imaging the cornea in clinical settings for humans have been developed (Cavanagh et al., 1993). As described above for visualizing the retina itself in live animals, minor modifications to such slit-lamp microscopes would allow application to small rodents such as the mouse. Others have used conventional fluorescent microscopes for visualizing sensory nerves of living mice, using non-specific dyes such as 4-D-2-ASP or FluoroGold® that simply outline structure but provide no information on gene expression (Harris et al., 1989; LaVail et al., 1993).

The retina and cornea are appealing sites as they are accessible with a minimum of intervening tissue. The cornea has long been used to test potential toxicity of ophthalmic medications, for obvious reasons, but also as a model for general cutaneous toxicity of any substance that would be applied to or might inadvertently come into contact with the skin, such as in the Draize test. Therefore, the retinal and corneal tissues serve as novel sites for evaluating neuronal toxicity, due to the relative accessibility for epifluorescent and confocal microscopes, as described above.

In vivo neurotoxicity of substances may be assayed by other methods as well. For example, the transgenic mice may be exposed to neurotoxic substances (Seki et al., 2006) at a predetermined dosages for predetermined periods of time. The mice may then be sacrificed and ocular tissues may be prepared as tissue sections or whole mounts for subsequent analysis by epifluorescence microscopy, confocal microscopy or fluorometry as described in the examples. Methods of quantitating the fluorescent signals generated by these assays are well known. It is preferable that these results be compared to those obtained from negative, vehicle- or sham treated control mice which have not been exposed to the candidate substance or potential ophthalmic agent.

### III. METHODS OF SCREENING AND/OR MANUFACTURING OPHTHALMIC AGENTS

The present invention contemplates methods for screening and/or manufacturing topical ophthalmic pharmaceutical agents that can be administered effectively and safely to treat corneal dry eye syndrome and retinal neuropathies associated with glaucoma and AMD. These methods may comprise the use of screening candidate substances by utilizing a non-human transgenic animal model expressing a fluorescent protein (FP). Other screening methods may also include in vitro or in cyto screening assays utilizing the cells isolated from the transgenic animals. Thus, the present invention can perform random screening of large libraries of candidate substances; alternatively, the methods may be used to focus on particular classes of compounds selected with the aim of finding structural attributes that are believed to make them more likely to have therapeutic efficacy and selectivity.

To identify, make, generate, provide, manufacture or obtain an ophthalmic pharmaceutical, one generally will determine the activity, for example, neuronal activity, neuronal sensitivity, neuronal degradation, in the presence, absence, or both of the candidate substance, wherein an ophthalmic pharmaceutical is defined as any substance that effectively and safely treats corneal inflammation, dry eye syndrome and/or retinal neuropathies associated with glaucoma and AMD. For example, a method may generally comprise:
(a) providing a non-human transgenic animal expressing a fluorescent protein in ocular tissue;
(b) providing a candidate substance;
(c) selecting the ophthalmic agent by assessing the ability of the candidate substance to provide protection to the ocular tissue; and
(e) manufacturing the agent.

The present invention particularly contemplates the use of various animal models. Transgenic mice may be produced by pronuclear injection as disclosed in U.S. Pat. Nos. 4,736,866, 5,625,125, 5,489,742, 5,583,009, 5,573,933 and 4,873,191, incorporated herein by reference. Transgenic animals, especially mice, may also be produced by homologous recombination or gene targeting in stem cells as disclosed in U.S. Pat. Nos. 5,614,396, 5,416,260 and 5,413,923, incorporated herein by reference.

Treatment of animals with test compounds involve the administration of the compound, in an appropriate form, to the animal. Administration is by any route that could be utilized for clinical or non-clinical purposes. Specifically contemplated are topical ophthalmic administration, intracameral injection and intravitreal injection.

In certain embodiments, the animal model is a non-human transgenic animal expressing FP in ocular tissues; more specifically, nervous ocular tissue can be used. The candidate substance is administered to the animal. Next, the ophthalmic agent is selected by assessing the effect of the candidate substance on the animal, for example, determining protection of the ocular tissue, efficacy in treatment of, for example, corneal inflammation, dry eye syndrome and/or retinal disorders. Upon identification of the agent, the method may further provide the step of manufacturing of the ophthalmic agent using well known techniques in the art, such as synthesizing the compound or deriving the compound from a natural source.

In certain embodiments, protection of ocular tissues can be determined by assessing known endpoints or parameters that one of skill in the art routinely uses. For example, to determine the protection of an agent for corneal dry eye syndrome, one can measure tear film secretion and tear film break up time, measure the chemical composition of tear film and perform ocular surface evaluation with fluorescein, lissamine green or rose bengal stain. Yet further, confocal scanning laser opthalmoscopy can also be used to assess the agents in vivo.

Regarding the determination of protection of an agent for retinal neuropathies associated with glaucoma and AMD, one can assess therapeutic efficacy for decreasing IOP, reducing the loss of retinal ganglion cells and the loss of optic nerve axons. In the past, methods to measure ganglion cell number involved the retrograde transport of fluorescent dye down the axons in the optic nerve from the initial injection site in the brain followed by counting of retinal ganglion cell bodies in fluorescent images of ex vivo retinal preparations (Selles-Navarro et al., 1996). Measurements of the optic nerve involved ex vivo counting of axon structures in sections of optic nerve (Inman et al., 2006). The present invention will perform these measurements in an in vivo examination of the eye, and thereby assess therapeutic efficacy.

In further embodiments, instead of determining protection of the agent, efficacy of the agent can be determined by measuring a decrease in one of the symptoms, for example, ameliorating at least one symptom of dry eye syndrome, glaucoma and/or AMD.

In addition to in vivo assays, in vitro or in cyto assays may also be used in the present invention. Cells that express GFP can be utilized for screening of candidate substances. For example, cells containing GFP proteins can be used to study various functional attributes of candidate compounds. In such assays, the candidate compound is formulated appropriately for the assay and contacted with a target cell.

Depending on the assay, culture may be required. The cell may then be examined by virtue of a number of different physiologic assays (e.g., growth, size, or survival). Alternatively, molecular analysis may be performed. This involves assays such as those for protein production, substrate utilization, mRNA expression (including differential display of whole cell or polyA RNA) and others.

It will, of course, be understood that all the screening methods of the present invention are useful in themselves notwithstanding the fact that effective candidates may not be found. The invention provides methods for screening for such candidates, not solely methods of finding them.

In an extension of any of the previously described screening assays, the present invention also provide for methods of producing or manufacturing ophthalmic agents. These methods comprise any of the preceding screening steps followed by an additional step of “producing or manufacturing the candidate substance identified as an ophthalmic agent”. Manufacturing can entail any well known and standard technique used by those of skill in the art, such as synthesizing the compound and/or deriving the compound from a natural source.

### IV. CANDIDATE SUBSTANCES

As used herein, the term “candidate substance” refers to any molecule that may potentially be a topical ophthalmic agent. Candidate compounds may include fragments or parts of naturally-occurring compounds or may be found as active combinations of known compounds which are otherwise inactive. The candidate substance can be a nucleic acid, a polypeptide, a small molecule, etc.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

A. Dry Eye

In certain embodiments, the candidate substance can be an inhibitor of cytokine synthesis, for example, inhibitors of MAP kinases (p38) include (5-(2-amino-4-pyrimidinyl)-4-(4-fluorophenyl)-1-(4-piperidinyl)imidazole) [“SB-220025”]; inhibitors of JNK include anthra[1,9-cd]pyrazol-6(2H)-one [“SP-600125”]; inhibitors of ICE include pralnacasan (HMR3480/VX-740); TNF mRNA translation inhibitors include (D)Arginyl-(D)Norleucyl-(D)Norleucyl-(D)Arginyl-(D)Norleucyl-(D)Norleucyl-(D)Norleucyl-Glycine-(D)Tyrosine-amide, acetate salt [“RDP58”]; NFkB inhibitors include 2-chloro-N-[3,5-di(trifluoromethyl)phenyl]-4-(trifluoromethyl)pyrimidine-5-carbo xamide [“SP-100030”], and triflusal; AP-1 inhibitors include SP-100030; and RXR agonists include bexarotene. Antioxidants and free radical scavenger compounds can also be candidate substances, such as those compounds disclosed in U.S. Pat. No. 5,846,988 to Hellberg entitled “Thiazolidine-4-carboxylic acid derivatives as cytoprotective agents”.

B. Retinal Neuropathies

In certain embodiments, the candidate substances can include substances known to reduce IOP, for example, beta-blockers, such as timolol and betaxolol, and carbonic anhydrase inhibitors, such as dorzolamide and brinzolamide. Other agents may also include prostaglandin analogs, which are believed to reduce intraocular pressure by increasing uveoscleral outflow. Three marketed prostaglandin analogs are latanoprost, bimatoprost and travoprost. Still further, other agents can reduce IOP include rho kinase inhibitors, α2 agonists, miotics, and serotonergic agonists. Neuroprotective agents may include trophic factors or peptido-mimetics of trophic factors such as brain-derived neurotrophic factor, BNDF, or glial derived neurotrophic factor, GNDF, pigment epithelial-derived factor, PEDF, ciliary neurotrophic factor, CNTF and any inducers of neuronal trophic factors. Other neuroprotective agents could include anti-apoptosis agents such as caspase inhibitors and kinase inhibitors, and anti-inflammatory agents such as anecortave acetate and superoxide dismutase mimetics.

Other agents that may be used to treat retinal diseases include anti-VEGF medications (i.e., pegaptanib (Macugen®), ranibizumab (Lucentis®), bevacizumab (Avastin®); and anti-inflammatories (i.e., steriods, for example, triamcinolone (Kenalog®).

In particular embodiments, the substance is a receptor tyrosine kinase (RTK) inhibitor. Exemplary receptor tyrosine kinase inhibitors include N-[4-[3-amino-1H-indazol-4-yl)phenyl]-N′-(2-fluoro-5-methylphenyl)urea; N-[4-(3-amino-1,2-benzisoxazol-4-yl)phenyl]-N′-(3-methylphenyl)urea; N-[4-(3-amino-1,2-benzisoxazol-4-yl)phenyl]-N′-[2-(trifluoromethyl)phenyl]urea; N-[4-(3-amino-1,2-benzisoxazol-4-yl)phenyl]-N′-(2-fluoro-5-methylphenyl)urea; N-[4-(3-amino-1,2-benzisoxazol-4-yl)phenyl]-N′-[3-(trifluoromethyl)phenyl]urea; N-[4-(3-amino-1,2-benzisoxazol-4-yl)phenyl]-N′-[2-fluoro-5-(trifluoromethyl)phenyl]urea; N-[4-(3-amino-7-methoxy-1,2-benzisoxazol-4-yl)phenyl]-N′-[2-fluoro-5-(trifluoromethyl)phenyl]urea; N-[4-(3-amino-7-methoxy-1,2-benzisoxazol-4-yl)phenyl]-N′-(3-methylphenyl)urea; N-[4-(3-amino-7-methoxy-1,2-benzisoxazol-4-yl)phenyl]-N′-[3-(trifluoromethyl)phenyl]urea; N-[4-(3-amino-7-methoxy-1,2-benzisoxazol-4-yl)phenyl]-N′-(3-chlorophenyl)-urea; N-[4-(3-amino-7-methoxy-1,2-benzisoxazol-4-yl)phenyl]-N′-(2-fluoro-5-methylphenyl)urea; N- {4-[3-amino-7-(4-morpholinylmethyl)-1,2-benzisoxazol-4-yl]phenyl}-N′-[2-fluoro-5-(trifluoromethyl)phenyl]urea; N-{4-[3-amino-7-(4-morpholinylmethyl)-1,2-benzisoxazol-4-yl]phenyl}-N′-[3-(trifluoromethyl)phenyl]urea; N-{4-[3-amino-7-(4-morpholinylmethyl)-1,2-benzisoxazol-4-yl]phenyl}-N′-(3-chlorophenyl)urea; N-{4-[3-amino-7-(4-morpholinylmethyl)-1,2-benzisoxazol-4-yl]phenyl}-N′-(3-methylphenyl)urea; N-{4-[3-amino-7-(4-morpholinylmethyl)-1,2-benzisoxazol-4-yl]phenyl}-N′-(2-fluoro-5-methylphenyl)urea; N-{4-[3-amino-7-(4-morpholinylmethyl)-1,2-benzisoxazol-4-yl]phenyl}-N′-(3-,5-dimethylphenyl)urea; N-{4-[3-amino-7-(4-morpholinylmethyl)-1,2-benzisoxazol-4-yl]phenyl}-N′-(3-phenoxyphenyl)urea; N-{4-[3-amino-7-(4-morpholinylmethyl)-1,2-benzisoxazol-4-yl]phenyl}-N′-(3-bromophenyl)urea; N-(4-{3-amino-7-[2-(4-morpholinyl)ethoxy]-1,2-benzisoxazol-4-yl}phenyl)-N-′-[3-(trifluoromethyl)phenyl]urea; N-(4-{3-amino-7-[2-(4-morpholinyl)ethoxy]-1,2-benzisoxazol-4-yl}phenyl)-N′-(2-fluoro-5-methylphenyl)urea; N-(4-{3-amino-7-[2-(4-morpholinyl)ethoxy]-1,2-benzisoxazol-4-yl}phenyl)-N′-[2-fluoro-5-(trifluoromethyl)phenyl]urea; N-(4- {3-amino-7-[2-(4-morpholinyl)ethoxy]-1,2-benzisoxazol-4-yl}phenyl)-N′-(3-methylphenyl)urea; N-[4-(3-amino-1,2-benzisoxazol-4-yl)phenyl]-N′-(3,5-dimethylphenyl)urea; N-[4-(3-amino-1,2-benzisoxazol-4-yl)phenyl]-N′-phenylurea; N-[4-(3-amino-1,2-benzisoxazol-4-yl)phenyl]-N′-(4-methylphenyl)urea; N-[4-(3-amino-1,2-benzisoxazol-4-yl)phenyl]-N′-(3-cyanophenyl)urea; N-[4-(3-amino-1,2-benzisoxazol-4-yl)phenyl]-N′-[4-fluoro-3-(trifluoromethyl)phenyl]urea; N-[4-(3-amino-1,2-benzisoxazol-4-yl)phenyl]-N′-(3-bromophenyl)urea; N-[4-(3-amino-1,2-benzisoxazol-4-yl)phenyl]-N′-(3-chlorophenyl)urea; N-[4-(3-amino-1,2-benzisoxazol-4-yl)phenyl]-N′-(3-ethylphenyl)urea; N-[4-(3-amino-1,2-benzisoxazol-4-yl)phenyl]-N′-[4-(trifluoromethyl)phenyl]urea; N-[4-(3-amino-1,2-benzisoxazol-4-yl)phenyl]-N′-(3-fluoro-4-methylphenyl)urea; N-[4-(3-amino-1,2-benzisoxazol-4-yl)phenyl]-N′-(3-fluorophenyl)-urea; N-[4-(3-amino-1,2-benzisoxazol-4-yl)phenyl]-N′-(3,5-difluorophenyl)urea; N-[4-(3-amino-1,2-benzisoxazol-4-yl)phenyl]-N′-(3-methoxyphenyl)urea; N-[4-(3-amino-1,2-benzisoxazol-4-yl)phenyl]-N′-(4-methoxyphenyl)urea; N-[4-(3-amino-1,2-benzisoxazol-4-yl)phenyl]urea; N-[4-(3-amino-1,2-benzisoxazol-4-yl) phenyl]-N′-(3-nitrophenyl)urea; N-[4-(3-amino-1,2-benzisoxazol-4-yl)phenyl]-N′-(4-fluorophenyl)urea; N-[4-(3-amino-1,2-benzisoxazol-4-yl)phenyl]-N′-(2-fluorophenyl)urea; N-[4-(3-amino-1,2-benzisoxazol-4-yl)phenyl]-N′-(3-chloro-4-fluorophenyl)urea; N-[4-(3-amino-1,2-benzisoxazol-4-yl)phenyl]-N′-(3-chloro-4-methoxyphenyl)urea; N-[4-(3-amino-1,2-benzisoxazol-4-yl)phenyl]-N′-[4-(dimethylamino)phenyl]urea; N-[4-(3-amino-1,2-benzisoxazol-4-yl)phenyl]-N′-[4-(trifluoromethoxy)phenyl]urea; N-[4-(3-amino-1,2-benzisoxazol-4-yl)phenyl]-N′-[2-(trifluoromethoxy)phenyl]urea; N-[4-(3-amino-1,2-benzisoxazol-4-yl)phenyl]-N′-[3,5-bis(trifluoromethyl)phenyl]urea; N-[4-(3-amino-1,2-benzisoxazol-4-yl)phenyl]-N′-(3-chloro-4-methylphenyl)urea; N-[4-(3-amino-7-methoxy-1,2-benzisoxazol-4-yl)phenyl]-N′-[3,5-bis(trifluoromethyl)phenyl]urea; N-[4-(3-amino-7-methoxy-1,2-benzisoxazol-4-yl)phenyl]-N′-[4-(trifluoromethoxy)phenyl]urea; N-[4-(3-amino-7-methoxy-1,2-benzisoxazol-4-yl)phenyl]-N′-(3-fluorophenyl)urea; N-[4-(3-amino-7-methoxy-1,2-benzisoxazol-4-yl)phenyl]-N′-(3-methoxyphenyl)urea; N-[4-(3-amino-7-methoxy-1,2-benzisoxazol-4-yl)phenyl]-N′-(3,5-difluorophenyl)urea; N-[4-(3-amino-7-methoxy-1,2-benzisoxazol-4-yl)phenyl]-N′-(4-methylphenyl)urea; N-[4-(3-amino-7-methoxy-1,2-benzisoxazol-4-yl) phenyl]-N′-(3-bromophenyl)urea; N-[4-(3-amino-7-methoxy-1,2-benzisoxazol-4-yl)phenyl]-N′-(3,5-dimethylphenyl)urea; N-[4-(3-amino-7-methoxy-1,2-benzisoxazol-4-yl)phenyl]-N′-[4-(dimethylamino)phenyl]urea; N-[4-(3-amino-7-methyl-1,2-benzisoxazol-4-yl)phenyl]-N′-(3-methylphenyl)urea; N-[4-(3-amino-7-methyl-1,2-benzisoxazol-4-yl)phenyl]-N′-(3-chlorophenyl)urea; N-[4-(3-amino-7-methyl-1,2-benzisoxazol-4-yl)phenyl]-N′-(2-fluoro-5-methylphenyl)urea; N-[4-(3-amino-7-methyl-1,2-benzisoxazol-4-yl)phenyl]-N′-[2-fluoro-5-(trifluoromethyl)phenyl]urea; N-[4-(3-amino-7-methyl-1,2-benzisoxazol-4-yl)phenyl]-N′-[3-(trifluoromethyl)phenyl]urea; N-[4-(3-amino-7-methyl-1,2-benzisoxazol-4-yl)phenyl]-N′-(3,5-dimethylphenyl)urea; N-[4-(3-amino-7-methyl-1,2-benzisoxazol-4-yl)phenyl]-N′-(3-ethylphenyl)urea; N-[4-(3-amino-7-methyl-1,2-benzisoxazol-4-yl)phenyl]-N′-(4-methylphenyl)urea; N-[4-(3-amino-7-methyl-1,2-benzisoxazol-4-yl)phenyl]-N′-[4-(trifluoromethoxy)phenyl]urea; N-[4-(3-amino-7-methyl-1,2-benzisoxazol-4-yl)phenyl]-N′-(3-fluoro-4-methylphenyl)urea; N-[4-(3-amino-7-methyl-1,2-benzisoxazol-4-yl)phenyl]-N′-(3-methoxyphenyl)urea; N-[4-(3-amino-7-methyl-1,2-benzisoxazol-4-yl)phenyl]-N′-phenylurea; N-[4-(3-amino-7-methyl-1,2-benzisoxazol-4-yl)phenyl]-N′-[3,5-bis(trifluoromethyl)phenyl]urea; N-[4-(3-amino-7-methyl-1,2-benzisoxazol-4-yl)phenyl]-N′-(3-bromophenyl)urea; N-[4-(3-amino-7-methyl-1,2-benzisoxazol-4-yl)phenyl]-N′-(3-fluorophenyl)urea; N-[4-(3-amino-7-methoxy-1,2-benzisoxazol-4-yl)phenyl]-N′-[4-fluoro-3-(trifluoromethyl)phenyl]urea; N-[4-(3-amino-7-methoxy-1,2-benzisoxazol-4-yl)phenyl]-N′-(4-fluoro-3-methylphenyl)urea; N-[4-(3-amino-7-fluoro-1,2-benzisoxazol-4-yl)phenyl]-N′-[3-(trifluoromethyl)phenyl]urea; N-[4-(3-amino-7-fluoro-1,2-benzisoxazol-4-yl)phenyl]-N′-(3-chlorophenyl)urea; N-[4-(3-amino-7-fluoro-1,2-benzisoxazol-4-yl)phenyl]-N′-[4-fluoro-3-(trifluoromethyl)phenyl]urea; N-[4-(3-amino-7-fluoro-1,2-benzisoxazol-4-yl)phenyl]-N′-(3-methylphenyl)urea; N-[4-(3-amino-7-fluoro-1,2-benzisoxazol-4-yl)phenyl]-N′-[2-fluoro-5-(trifluoromethyl)phenyl]urea; N-[4-(3-amino-7-fluoro-1,2-benzisoxazol-4-yl)phenyl]-N′-(2-fluoro-5-methylphenyl)urea; N-{4-[3-amino-7-(trifluoromethoxy)-1,2-benzisoxazol-4-yl]phenyl}-N′-[2-fluoro-5-(trifluoromethyl)phenyl]urea; N-{4-[3-amino-7-(trifluoromethoxy)-1,2-benzisoxazol-4-yl]phenyl}-N′-[3-(trifluoromethyl)phenyl]urea; N-{4-[3-amino-7-(trifluoromethoxy)-1,2-benzisoxazol-4-yl]phenyl}-N′-(2-fluoro-5-methylphenyl)urea; N-{4-[3-amino-7-(trifluoromethoxy)-1,2-benzisoxazol-4-yl]phenyl}-N′-(3-chlorophenyl)urea; N-{4-[3-amino-7-(trifluoromethoxy)-1,2-benzisoxazol-4-yl]phenyl}-N′-(3-bromophenyl)urea; N-{4-[3-amino-7-(trifluoromethoxy)-1,2-benzisoxazol-4-yl]phenyl}-N′-[4-fluoro-3-(trifluoromethyl)phenyl]urea; and N-{4-[3-amino-7-(trifluoromethoxy)-1,2-benzisoxazol-4-yl]phenyl}-N′-(4-fluoro-3-methylphenyl)urea.

In particular embodiments, the receptor tyrosine kinase inhibitor is N-[4-[3-amino-1H-indazol-4-yl]phenyl]-N′-(2-fluoro-5-methylphenyl)urea. Detailed information regarding RTK inhibitors can be found in U.S. Patent App. Pub. No. 2006/0189608, hereby specifically incorporated by reference.

### V. TREATMENT

In certain aspects of the present invention, compounds are used to treat and/or prevent dry eyes and associated corneal inflammation. Other aspects of the present invention can involve the use of the identified agent to treat retinal diseases.

A. Corneal Inflammation and/or Dry Eye Syndrome

Inflammation of the cornea commonly presents as a painful red eye with reduced visual acuity due to cellular infiltration and later, corneal edema. Dry eye disorder or dry eye syndrome can be caused from decreased production of fluids from the tear glands and/or an imbalance in the formulation or composition of the produced tears (e.g., decreased lipid secretion from Meibomian glands, or changes in lipid content). Thus, the agents identified in the present invention can be used to treat corneal inflammation or dry eye or dry eye syndrome in at least one eye of a subject.

Treatment and/or prevention methods involve treating an individual with an effective amount of a topical ophthalmic agent identified using the present invention. An effective amount is described, generally, as that amount sufficient to detectably and repeatedly to ameliorate, reduce, minimize or limit the extent of a disease or its symptoms. More specifically, it is envisioned that the treatment with ophthalmic agent thereof will stabilize or improve visual function (as measured by visual acuity, visual field, or other method known to those of ordinary skill in the art), decrease eye burning and inflammation, decrease eye irritation, decrease eye redness, decrease symptoms of dry eye including inflammation, and/or increase tear production.

The subject to be treated is a mammal, preferably a human. In certain embodiments, a subject can be a subject who is suffering from dry eye syndrome or corneal inflammation. In some embodiments, the subject is a subject at risk of developing dry eye or dry eye syndrome. Thus, in certain embodiments of the invention, methods include identifying a subject in need of treatment. For example, a subject that is susceptible or at risk for developing dry eyes may be a subject suffering from menopause, a subject that lives in a hot, dry or windy climate, a subject exposed to high altitudes, a subject that spends excessive amounts of time reading and/or working on a computer, a subject that wears contact lens, a subject having undergone surgery (e.g., LASIK), a subject exposed to air conditioning and/or heating, a subject suffering from thyroid conditions, vitamin A deficiency, Parkinson's, diabetes, rheumatoid arthritis, SLE, and/or Sjogren's, a subject suffering from eyelid disorders (e.g., blepharitis, Bell's palsy, facial palsy, Grave's disease) and a subject suffering from side effects from drugs, for example antihistamines, decongestants, antidepressants, diuretics, beta blockers, oral contraceptives, opiate-based drugs, THC-based drugs.

B. Retinal Neuropathy and/or Optic Nerve Damage

In certain aspects of the present invention, ophthalmic agents identified using the present invention can be used to treat and/or prevent retinal neuropathy and optic nerve damage.

Types of optic nerve damage that may be treated and/or prevented using the agents identified in the present invention can include, for example, glaucoma and other optic neuropathies. Glaucoma more specifically includes primary open angle glaucoma, acute angle closure glaucoma, normal tension glaucoma, low tension glaucoma, ocular hypertension. Optic neuropathies that may be treated and/or prevented by the present invention may include for example, ischemic optic neuropathies, such as anterior ischemic optic neuropathy and optic neuropathies associated with vascular disease such as diabetes. Yet further, another optic neuropathy that may be included is Leber's hereditary optic neuropathy.

Treatment and/or prevention methods in certain embodiments will involve treating an individual with an effective amount of an ophthalmic agent, preferably in a topical formulation identified using the present invention. In general, it is contemplated that the agent will be neuroprotective. An effective amount is described, generally, as that amount sufficient to detectably and repeatedly ameliorate, reduce, minimize or limit the extent of a disease or its symptoms. It is envisioned that the treatment with the ophthalmic agent identified by the present invention will decrease the intraocular pressure, increase visual function, decrease retina deterioration, decrease the severity of glaucoma, and/or delay or prevent the onset of optic nerve damage resulting in glaucoma, and/or delay or prevent the onset of macular degeneration.

A subject can be an individual who is known or suspected of being free of a particular disease or health-related condition at the time the relevant agent is administered. The individual, for example, can be a subject with no known disease or health-related condition (i.e., a healthy subject). In some embodiments, the individual is a subject at risk of developing a particular disease or health-related condition.

### VI. PHARMACEUTICS AND FORMULATIONS

Regarding the methods set forth herein, the ophthalmic agent(s) identified in the present invention can be formulated in any manner known to those of ordinary skill in the art.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of the ophthalmic agent(s) in a composition and appropriate dose(s) for the individual subject.

In certain non-limiting embodiments, the pharmaceutical compositions of the present invention may comprise, for example, at least about 0.1%, by weight or volume, of a ophthalmic agent. In other embodiments, an ophthalmic agent may comprise between about 2% to about 75% of the weight or volume of the unit, or between about 25% to about 60%, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient.

In particular embodiments, the compositions are suitable for application to mammalian eyes. For example, for ophthalmic administration, the formulation may be a solution, a suspension, a gel, or an ointment.

In preferred aspects, the topical ophthalmic compositions are formulated for aqueous solution in the form of drops. The term “aqueous” typically denotes an aqueous composition wherein the carrier is to an extent of >50%, more preferably >75% and in particular >90% by weight water. These drops may be delivered from a single dose ampoule which may preferably be sterile and thus rendering bacteriostatic components of the formulation unnecessary. Alternatively, the drops may be delivered from a multi-dose bottle which may preferably comprise a device which extracts preservative from the formulation as it is delivered, such devices being known in the art.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of one or more proteasome inhibitors. Examples or artificial tears compositions useful as artificial tears carriers include, but are not limited to, commercial products, such as Tears Naturale®, Tears Naturale II®, Tears Naturale Free®, and Bion Tears® (Alcon Laboratories, Inc., Fort Worth, Tex.). Examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Glonek et al.); the foregoing patents are incorporated herein by reference to the extent they disclose phospholipid compositions useful as phospholipid carriers of the present invention. In other aspects, components of the invention may be delivered to the eye as a concentrated gel or similar vehicle which forms dissolvable inserts that are placed beneath the eyelids.

The compositions of the present invention may also contain a surfactant. Various surfactants useful in topical ophthalmic formulations may be employed. The surfactant(s) may provide additional chemical stabilization of the compositions and may further provide for physical stability. In other words, the surfactants may aid in preventing chemical degradation of the compositions and also prevent the compounds from binding to the containers in which their compositions are packaged. As used herein, “an effective concentration of surfactant(s)” refers to a concentration that enhances the chemical and physical stability of the compositions. Examples of surfactants include, but are not limited to: Cremophor® EL, polyoxyl 20 ceto stearyl ether, polyoxyl 40 hydrogenated castor oil, polyoxyl 23 lauryl ether and poloxamer 407 may be used in the compositions. A preferred surfactant is polyoxyl 40 stearate. The concentration of surfactant will vary, depending on the concentration of the composition and optional ethanol present. In general, however, the surfactant(s) concentration will be about 0.001 to 2.0% w/v. Preferred compositions of the present invention will contain about 0.1% w/v of polyoxyl 40 stearate.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Induction of Inflammation

The ability of certain agents to induce corneal inflammation can be evaluated by in vivo assays using the thy1-FP transgenic mice.

Both eyes of mice are topically dosed with μL aliquot of a test compound in a vehicle. Animals are monitored continuously for 0.5 hr post-dose and then every 0.5 hours through 2 hours or until inflammation is present.

### Example 2

### Ocular Safety Evaluation

The ability of certain agents to safely lower IOP may be evaluated by in vivo assays using the thy1-GFP transgenic mice or other transgenic or non-transgenic mice.

Both eyes of mice are topically dosed with μL aliquot of a test compound in a vehicle. Animals are monitored continuously for 0.5 hr post-dose and then every 0.5 hours through 2 hours or until effects are no longer evident. Animals can be monitored for longer or shorter time periods to determine ocular toxicity.

### Example 3

### Acute IOP Response

Intraocular pressure (IOP) is determined with a Mentor Classic 30 pneumatonometer after light corneal anesthesia with 0.1% proparacaine. Eyes of thy1-FP transgenic mice are rinsed with one or two drops of saline after each measurement. After a baseline IOP measurement, test compound is instilled in μL aliquots to one or both eye of each animal or compound to one eye and vehicle to the contralateral eye. Subsequent IOP measurements are taken at 0.5, 1, 2, 3, 4, and 5 hours.

### Example 4

### Preparation of Corneal and Retinal Tissue

Four corneas were harvested from total of two adult thy1-GFP transgenic mice (3-month old; mice line Feng-21). The thy1-GFP transgenic mice were anesthetized and perfused through the heart, first with lactated Ringer's solution, then with 4% paraformaldehyde for 30 minutes. After the eyes were enucleated and placed in the ice-cold PBS buffer. Eye globes were cut along the opaque sclera close to the corneoscleral limbus. The lens and iris were removed and discarded. Then, the cornea was flattened by four radial cuts. Corneal flat mount was mounted on a microscope slide, endothelial side up, for examination under a Nikon fluorescence con-focal microscope.

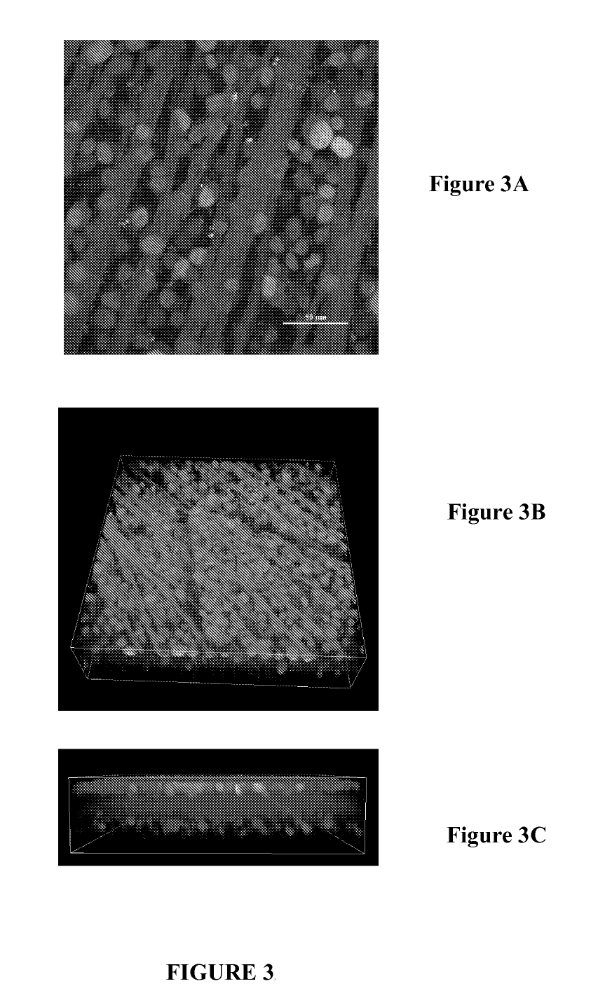

Corneal and retinal flat mounts were prepared from the eyes and examined with a Nikon confocal light microscope to verify cellular expression patterns. As shown in FIG. 3, both retinal ganglion cell bodies and nerve fibers are readily visible (A). The view in FIG. 3A is a two dimension maximum project view of stack of images collected from the retinal surface down to the start of the amacrine layer. The level of GFP expression in the amacrine layer is being assessed in frozen sections. Expression of GFP in the retinal ganglion cells appears to be highest in the nucleus (but not the nucleolus) and this is probably due to non-specific binding of GFP to the highly charged nuclear protein. Nerve fibers appear to be uniformly labeled as well. In a more three dimensional view, (B), it is clear the cells of the vasculature such as endothelium, pericytes or smooth muscle do not express GFP. Cross section representations, (C) reveal interesting processes between the RGC and amacrine cells.

### Example 5

### Laser Confocal Microscopy to Measure the Efficacy of a Test Compound

Thy1 transgenic mice are used for confocal analyses. Nontransgenic littermates (age- and sex-matched) are used as negative controls. Transgenic animals are administered the test compound. Positive controls are transgenic animals (age- and sex-matched) that are only administered a vehicle (no test compound), such as saline. Animals are euthanized and tissues of interest are isolated.

Tissues subjected to examination include the optic nerve, retina, and corneal tissue. Whole mounts of intact nerves or retina or cornea, are mounted in a perfusion chamber supplied with Ringer's physiological solution at room temperature for observation. Tissue samples are analyzed using a confocal laser scanning microscope system. For GFP imaging, filters are employed to provide excitation at 488 nm, detecting emission at wavelengths greater than 515 nm. The GFP can also be viewed using an ordinary epifluorescence microscope equipped with a filter set for fluorescein.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Pat. No. 4,873,191
U.S. Pat. No. 4,736,866
U.S. Pat. No. 5,413,923
U.S. Pat. No. 5,416,260
U.S. Pat. No. 5,489,742
U.S. Pat. No. 5,573,933
U.S. Pat. No. 5,583,009
U.S. Pat. No. 5,614,396
U.S. Pat. No. 5,625,125
Amsterdam et al., Dev. Biol., Vol. 171:123, 1995
Aoki et al., FEBS Letters, Vol. 384:193, 1996
Asbell et al., The Ocular Surface, Vol. 3(1):27-40, 2005 Ethis Comm, Inc., 2005
Barabino et al., IOVS, Vol. 45(6):1641-1646, June 2004
Barabino et al., Experimental Eye Research, Vol. 79(5):613-621
Caroni J., Neuroscience Methods, Vol. 71:3-9, 1997
Cavanagh et al., Ophthalmology, Vol. 100: 1444, 1993
Chalfie et al., Science, Vol. 263:802, 1994
Cousins et al., IOVS, Vol. 44(3):1221-1229, March 2003
De Paiva et al., IOVS, Vol. 47(7):2847-2856, July 2006
Espinosa-Heidmann et al., IOVS, Vol. 47(2):729-37, February 2006
Feng et al., Neuron, Vol. 28:41-51, 2000
Han et al., Korean Journal of Ophthalmology, Vol. 19(3):219-26, September 2005
Harris et al. J. Neuroscience, Vol. 9:2210, 1989
Higashide et al., IOVS, Vol. 47(7):2943-2950, July 2006
Inman et al., IOVS, Vol. 47(3):986-996, March 2006
Jabs et al., IOVS, Vol. 41:826-831, 2000
Ji et al., Vision Research, Vol. 45(2):169-179, January 2005
John et al., IOVS, Vol. 39(6):951-962, May 1998
Kawaguchi et al., IOVS, Vol. 47(7):2911-2916, July 2006
Kelley et al., Brain Research/Mol. Brain Research, Vol. 24:261-274, 1994
Labbe et al, J Ocul Pharmacol Ther., Vol. 22(4):267-78, August 2006
LaVail et al., Brain Research, Vol. 625:57, 1993
LaVail et al., Brain Research, Vol. 625:57-62, 1993
Marshall et al., Neuron, Vol. 14:211, 1995
Morris, Neuroscience, Vol. 7:133-160, 1985
Morrison et al., Experimental Eye Research, Vol. 64:85-96, 1997
Nakazawa et al., Molecular Vision, Vol. 11:1173-1182, 2005
Naumann et al., J Neuroscience Methods, Vol. 103(1):11-21, November 2000
Naumann et al., J Neuroscience Methods, Vol. 103(1):11-21, November 2000
Niswender et al., J. Microscopy, Vol. 180:109, 1995
Peters et al., Dev. Biol., Vol. 171:252, 1995
Robinson et al., Arthritis Rheum., Vol. 41(1):150-156, January 1998
Sabel et al., Nature Medicine, Vol. 3:244, 1997
Seki et al, Ann. N.Y. Acad. Science., Vol. 1070:531-534, 2006
Selles-Navarro et al., IOVS, Vol. 37(10):2002-2014, September 1996
Solomon et al., The Ocular Surface, Vol. 2:34-44, 2004 Ethis Comm., Inc. 2005
Takahashi et al., Clin Exp Immunol., Vol. 109:555-561, 1997
Urcola et al., Experimental Eye Research, Vol. 83(2):429-437, August 2006
Van Blokland et al., Clin Immunol., Vol. 103:111-124, 2002
Vidal et al., EMBO J., Vol. 9:833-840, 1990.
Wang et al., Nature, Vol. 369:400, 1994
Zhivov et al., In vivo Confocal Microscopy of the Ocular Surface The Ocular Surface, Vol. 4:81-93, 2006

What is claimed is:

1. A method of manufacturing a topical ophthalmic agent comprising the steps of:
(a) providing a non-human transgenic animal that selectively expresses a fluorescent protein (FP) in ocular tissue;
(b) providing a candidate substance suspected of providing protection to ocular nervous tissue;
(c) administering the candidate substance to the animal;
(d) selecting the agent by assessing the ability of the candidate substance to provide protection in ocular nervous tissue; and
(e) manufacturing the selected ophthalmic agent.

2. The method of claim 1, wherein the FP is green fluorescent protein (GFP).

3. The method of claim 1, wherein the FP is yellow fluorescent protein (YFP).

4. The method of claim 1, wherein the FP expression is driven by a neuron-specific promoter.

5. The method of claim 4, wherein the neuron-specific promoter is Thy-1.

6. The method of claim 1, wherein the ocular nervous tissue is corneal tissue or retinal tissue.

7. The method of claim 1, wherein protection is measured by a decrease in the degeneration of ocular related neurons.

8. The method of claim 7, wherein the neurons are retinal ganglion or optic nerve axons.

9. The method of claim 1, wherein protection is measured by an increase in tear film secretion.

10. A method of treating a subject suffering from or suspected of suffering from glaucoma comprising administering an effective amount of the agent identified in claim 1.

11. A method of treating a subject suffering from or suspected of suffering from age-related macular degeneration comprising administering an effective amount of the agent identified in claim 1.

12. A method of treating a subject suffering from or suspected of suffering from dry eye syndrome in at least one eye comprising administering an effective amount of the agent identified in claim 1.

13. A method of screening an agent for its efficacy in ameliorating the symptoms of dry eye syndrome, comprising administering a candidate agent to a non-human transgenic animal expressing a fluorescent protein product, and comparing the symptoms of dry eye syndrome in the non-human transgenic animal to one or more control animals, wherein a decrease in symptoms of dry eye syndrome in the animal treated with the test agent indicates efficacy of the agent.

14. A method of treating a subject suffering from dry eye syndrome comprising administering the agent identified in claim 13.

15. The method of claim 14, wherein said dry eye syndrome is in at least one eye of the subject.

16. A method of treating a subject suspected of suffering from dry eye syndrome comprising administering the agent identified in claim 13.

17. A method of screening an agent for its efficacy in ameliorating the symptoms of glaucoma, comprising administering a candidate agent to a non-human transgenic animal expressing a fluorescent protein product, and comparing the symptoms of glaucoma in the non-human transgenic animal to one or more control animals, wherein a decrease in symptoms of glaucoma in the animal treated with the test agent indicates efficacy of the agent.

18. A method of treating a subject suspect of suffering from glaucoma comprising administering the agent identified in claim 17.
